(19) United States
(12) Patent Application Publication
(10) Pub. No.: US 2017/0319236 A1
(43) Pub. Date: Nov. 9, 2017

(54) FLAT TOP STABILIZATION ROD FOR SPINAL AND OTHER SURGICAL PROCEDURES

(71) Applicants: James SPITLER, Boca Raton, FL (US); Peter HARRIS, Boca Raton, FL (US)

(72) Inventors: James SPITLER, Boca Raton, FL (US); Peter HARRIS, Boca Raton, FL (US)

(73) Assignee: FloSpine LLC, Boca Raton, FL (US)

(21) Appl. No.: 15/585,822

(22) Filed: May 3, 2017

Related U.S. Application Data
(60) Provisional application No. 62/330,891, filed on May 3, 2016.

(51) Int. Cl. A61B 17/70; A61B 17/86

(52) U.S. Cl. CPC A61B 17/701; A61B 17/8605; A61B 17/7032; A61B 17/7043

(57) ABSTRACT

The present invention addresses rod rotation and slippage inside the tulip rod channel(s), as well as forcible rod bending as the spine and musculature bias towards a deformity, for example. The present invention provides a stabilization rod that, fundamentally, includes at least one flat surface on at least one side of the construct, this flat surface engaging a corresponding flat surface associated with the head body, set screw, or locking cap. Multiple flat surfaces may, of course, be used. The stabilization rod may also include one or more ridges and/or one or more keyed channels that is/are engaged by the head body, set screw, or locking cap.

### CROSS-REFERENCE TO RELATED APPLICATION

The present patent application/patent claims the benefit of priority of co-pending U.S. Provisional Patent Application No. 62/330,891, filed on May 3, 2016, and entitled “FLAT TOP STABILIZATION ROD FOR SPINAL AND OTHER SURGICAL PROCEDURES,” the contents of which are incorporated in full by reference herein.

### FIELD OF THE INVENTION

The present invention relates generally to mechanical components for spinal and other surgical procedures. More specifically, the present invention relates to a flat top stabilization rod for spinal and other surgical procedures.

### BACKGROUND OF THE INVENTION

A variety of conventional pedicle screws, for example, are known to those of ordinary skill in the art. These pedicle screws typically consist of a threaded screw portion including an enlarged head end and a head body or “tulip.” The threaded screw portion engages a pedicle of a vertebra of a spine, and the head body correspondingly engages the enlarged head end of the threaded screw portion, optionally by passing the threaded screw portion down and through a hole manufactured into the bottom of the head body until the enlarged head end or the threaded screw portion is seated in a bottom portion of the head body. The pedicle screws can be monoaxial or polyaxial, allowing the head body multiple degrees of freedom with respect to the threaded screw portion, at least initially. Once placed, stabilization rods are inserted into the head bodies of adjacent pedicle screws and set screws or “locking caps” are placed to simultaneously secure the head bodies to their respective threaded screw portions and to lock the stabilization rods in place. A rigid framework is thereby formed, stabilizing and fixing the vertebrae of the spine. Similar assemblies are used for other anatomical structures.

Disadvantageously, however, these stabilization rods may experience segmental rotational prolapse following final tightening, essentially rotating inside the tulip rod channel(s), thereby compromising the rigid framework formed post implantation.

### BRIEF SUMMARY OF THE INVENTION

Accordingly, in various exemplary embodiments, the present invention addresses rod rotation and slippage inside the tulip rod channel(s), as well as forcible rod bending as the spine and musculature bias towards a deformity, for example. The present invention provides a stabilization rod that, fundamentally, includes at least one flat surface on at least one side of the construct, this flat surface engaging a corresponding flat surface associated with the head body, set screw, or locking cap. Multiple flat surfaces may, of course, be used. The stabilization rod may also include one or more ridges and/or one or more keyed channels that is/are engaged by the head body, set screw, or locking cap. For example, a 5.5 or 6-mm CoCr stabilization rod such as this provides additional strength against rod flattening and warping, and the flat/keyed surface greatly increases the resistance to rotation inside the tulip rod channel, thereby fully addressing unwanted rotation regression.

In one exemplary embodiment, the present invention provides a stabilization rod for coupling a plurality of bone anchors together, the stabilization rod comprising: an elongate body; wherein the elongate body comprises at least one flat surface; and wherein the at least one flat surface is configured and positioned to engage at least one corresponding flat surface associated with one or more of a head body assembly, a set screw, and a locking cap of the bone anchor such that relative rotation of the elongate body and the bone anchor is prevented. Optionally, the elongate body has a substantially circular cross-sectional shape truncated by the at least one flat surface. Alternatively, the elongate body has a substantially U-shaped cross-sectional shape truncated by the at least one flat surface. Optionally, the elongate body further comprises one or more longitudinal channels manufactured along the at least one flat surface and the one or more of the head body assembly, the set screw, and the locking cap comprise one or more protrusions that are configured to engage the one or more longitudinal channels manufactured along the at least one flat surface such that relative rotation of the elongate body and the bone anchor is prevented. Optionally, the elongate body further comprises one or more longitudinal ridges manufactured along the at least one flat surface and the one or more of the head body assembly, the set screw, and the locking cap comprise one or more recesses that are configured to engage the one or more longitudinal ridges manufactured along the at least one flat surface such that relative rotation of the elongate body and the bone anchor is prevented.

In another exemplary embodiment, the present invention provides a method for providing a stabilization rod for coupling a plurality of bone anchors together, the method comprising: providing an elongate body; wherein the elongate body comprises at least one flat surface; and wherein the at least one flat surface is configured and positioned to engage at least one corresponding flat surface associated with one or more of a head body assembly, a set screw, and a locking cap of the bone anchor such that relative rotation of the elongate body and the bone anchor is prevented; and disposing the elongate body in the head body. Optionally, the elongate body has a substantially circular cross-sectional shape truncated by the at least one flat surface. Alternatively, the elongate body has a substantially U-shaped cross-sectional shape truncated by the at least one flat surface. Optionally, the elongate body further comprises one or more longitudinal channels manufactured along the at least one flat surface and the one or more of the head body assembly, the set screw, and the locking cap comprise one or more protrusions that are configured to engage the one or more longitudinal channels manufactured along the at least one flat surface such that relative rotation of the elongate body and the bone anchor is prevented. Optionally, the elongate body further comprises one or more longitudinal ridges manufactured along the at least one flat surface and the one or more of the head body assembly, the set screw, and the locking cap comprise one or more recesses that are configured to engage the one or more longitudinal ridges manufactured along the at least one flat surface such that relative rotation of the elongate body and the bone anchor is prevented.

In a further exemplary embodiment, the present invention provides a bone anchor, comprising: a threaded screw portion; a head body coupled to the threaded screw portion; and an elongate body coupled to the head body; wherein the elongate body comprises at least one flat surface; and wherein the at least one flat surface is configured and positioned to engage at least one corresponding flat surface associated with one or more of the head body assembly, a set screw, and a locking cap of the bone anchor such that relative rotation of the elongate body and the head body is prevented. Optionally, the elongate body has a substantially circular cross-sectional shape truncated by the at least one flat surface. Alternatively, the elongate body has a substantially U-shaped cross-sectional shape truncated by the at least one flat surface. Optionally, the elongate body further comprises one or more longitudinal channels manufactured along the at least one flat surface and the one or more of the head body assembly, the set screw, and the locking cap comprise one or more protrusions that are configured to engage the one or more longitudinal channels manufactured along the at least one flat surface such that relative rotation of the elongate body and the bone anchor is prevented. Optionally, the elongate body further comprises one or more longitudinal ridges manufactured along the at least one flat surface and the one or more of the head body assembly, the set screw, and the locking cap comprise one or more recesses that are configured to engage the one or more longitudinal ridges manufactured along the at least one flat surface such that relative rotation of the elongate body and the bone anchor is prevented.

### BRIEF DESCRIPTION OF THE DRAWINGS

The present invention is illustrated and described herein with reference to the various drawings, in which like reference numbers are used to denote like assembly components/method steps, as appropriate, and in which:

FIG. 1 is a side perspective view of one exemplary embodiment of the flat top stabilization rod of the present invention, utilizing one flat surface engaging the locking screw (left), as compared to a conventional round stabilization rod (right)—note, all pedicle screws shown are monoaxial for simplicity only;

FIG. 2 is a perspective view of another exemplary embodiment of the flat top stabilization rod of the present invention, utilizing one flat surface, one central groove, and a pair of small raised outboard ridges engaging the locking screw or cap (left), as compared to a conventional round stabilization rod (right)—note, all pedicle screws shown are monoaxial for simplicity only;

FIG. 3 is a side perspective view of a further exemplary embodiment of the flat top stabilization rod of the present invention, utilizing a U-shape, one flat surface, and pair of raised inboard ridges engaging the locking screw or cap (left) and one flat surface and a pair of small raised outboard ridges engaging the locking screw or cap (center), as compared to a conventional round stabilization rod (right)—note, all pedicle screws shown are monoaxial for simplicity only;

FIG. 4 is a perspective view of a still further exemplary embodiment of the flat top stabilization rod of the present invention, utilizing a U-shape, one flat surface, and pair of raised inboard ridges engaging the locking screw or cap (left) and one flat surface, one central groove, and a pair of small raised outboard ridges engaging the locking screw or cap (center), as compared to a conventional round stabilization rod (right)—note, all pedicle screws shown are monoaxial for simplicity only; and

FIG. 5 is a perspective view of a still further exemplary embodiment of the flat top stabilization rod of the present invention, utilizing a U-shape, one flat surface, and pair of raised inboard ridges.

### DETAILED DESCRIPTION OF THE INVENTION

Again, in various exemplary embodiments, the present invention addresses rod rotation and slippage inside the tulip rod channel(s), as well as forcible rod bending as the spine and musculature bias towards a deformity, for example. The present invention provides a stabilization rod that, fundamentally, includes at least one flat surface on at least one side of the construct, this flat surface engaging a corresponding flat surface associated with the head body, set screw, or locking cap. Multiple flat surfaces may, of course, be used. The stabilization rod may also include one or more ridges and/or one or more keyed channels that is/are engaged by the head body, set screw, or locking cap. For example, a 5.5 or 6-mm CoCr stabilization rod such as this provides additional strength against rod flattening and warping, and the flat/keyed surface greatly increases the resistance to rotation inside the tulip rod channel, thereby fully addressing unwanted rotation regression.

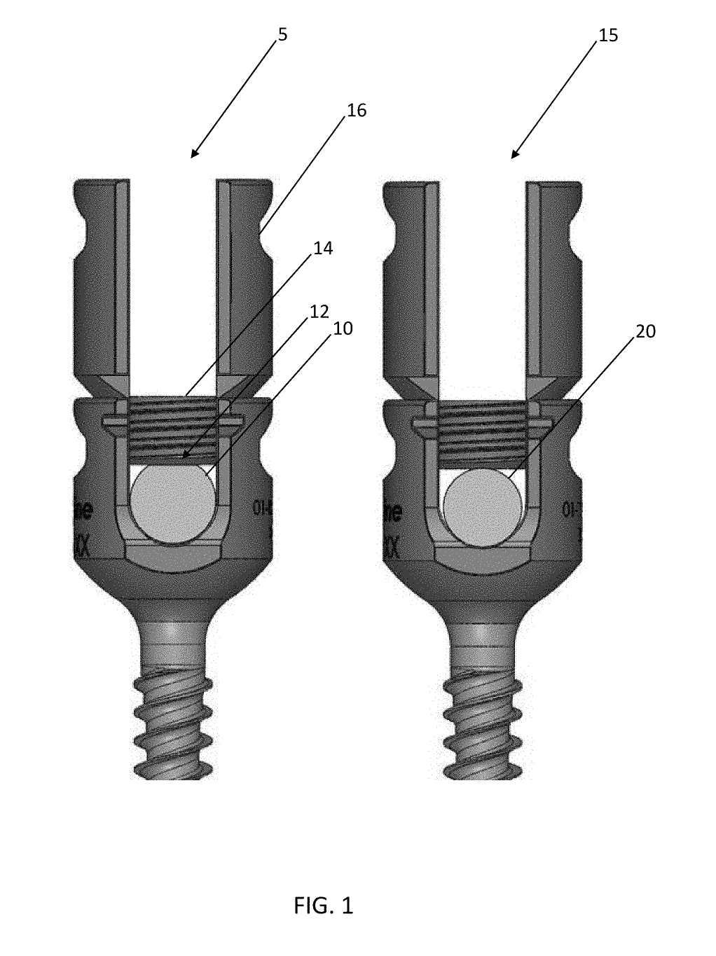

FIG. 1 is a side perspective view of one exemplary embodiment of the flat top stabilization rod 10 of the present invention, utilizing one flat surface 12 engaging the locking screw 14 (left), as compared to a conventional round stabilization rod 20 (right)—note, all pedicle screws 5 and 15 shown are monoaxial for simplicity only. In this exemplary embodiment, the stabilization rod 10 is an elongate structure that has a primarily circular cross-sectional shape, aside from the flat surface 12, where the circular cross-sectional shape is truncated. Preferably, the stabilization rod 10 is manufactured from a rigid or semi-rigid metallic or plastic material that is surgically compatible. The stabilization rod 10 has a minor dimension on the order to 5-10 mm and can be as long as is needed to span adjacent bone anchors in a given application. In the exemplary embodiment, the flat surface 12 engages the flat bottom of the locking screw 14 as it is driven into the head body 16, thereby securing the stabilization rod 10 and preventing rotation of the stabilization rod 10 within the head body 16. Multiple flat surfaces 12 can also be used and may engage other flat surfaces of or within the head body 16.

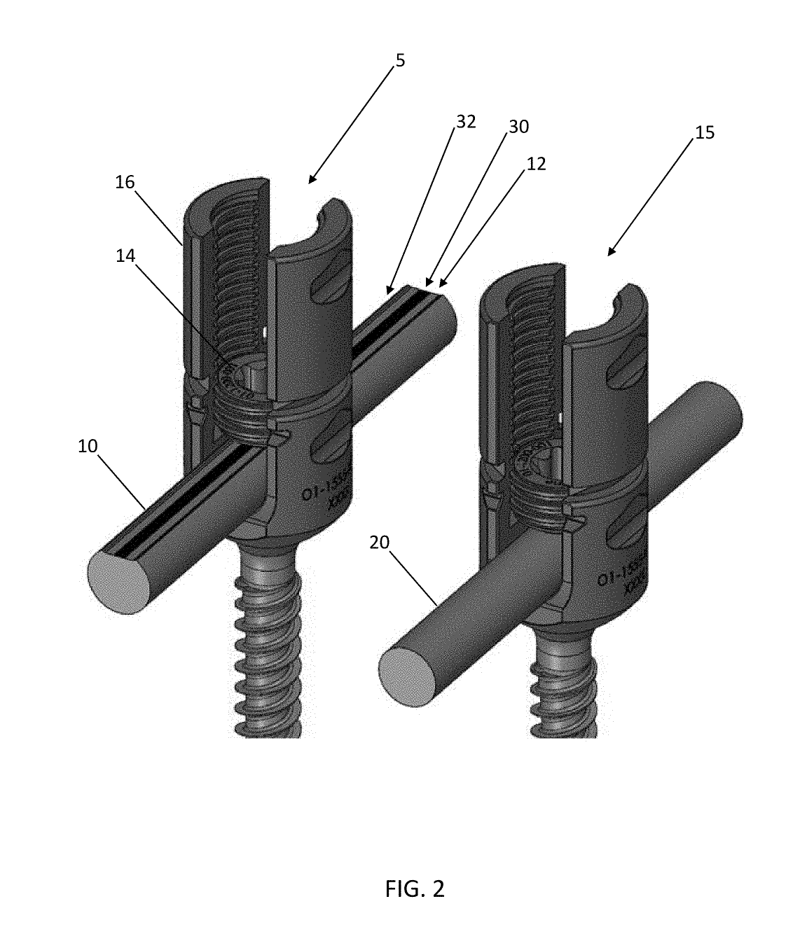

FIG. 2 is a perspective view of another exemplary embodiment of the flat top stabilization rod 10 of the present invention, utilizing one flat surface 12, one central groove 30, and a pair of small raised outboard ridges 32 engaging the locking screw or cap 14 (left), as compared to a conventional round stabilization rod 20 (right)—note, all pedicle screws 5 and 15 shown are monoaxial for simplicity only. In this exemplary embodiment, the stabilization rod 10 is an elongate structure that has a primarily circular cross-sectional shape, aside from the flat surface 12, central groove 30, and ridges 32, where the circular cross-sectional shape is truncated. Preferably, the stabilization rod 10 is again manufactured from a rigid or semi-rigid metallic or plastic material that is surgically compatible. The stabilization rod 10 has a minor dimension on the order to 5-10 mm and can be as long as is needed to span adjacent bone anchors in a given application. In the exemplary embodiment, the flat surface 12 engages the flat bottom of the locking screw or cap 14 as it is driven into the head body 16, thereby securing the stabilization rod 10 and preventing rotation of the stabilization rod 10 within the head body 16. Multiple flat surfaces 12 can also be used and may engage other flat surfaces of or within the head body 16. Optionally, the locking screw or cap 14, or other mating surface(s), may include corresponding ridges and/or grooves that engage the longitudinal central groove 30 and/or ridges 32 of the stabilization rod 10, thereby further securing the stabilization rod 10 with the head body 16.

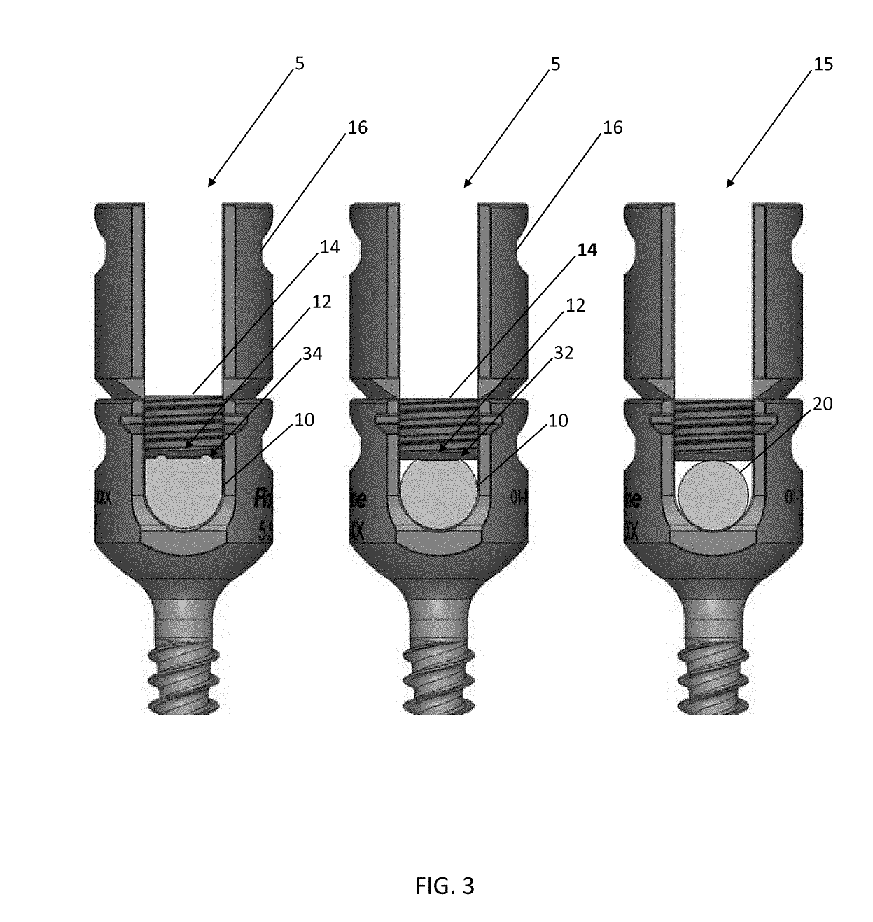

FIG. 3 is a side perspective view of a further exemplary embodiment of the flat top stabilization rod 10 of the present invention, utilizing a U-shape, one flat surface 12, and pair of raised inboard ridges 34 engaging the locking screw or cap 14 (left) and one flat surface 12 and a pair of small raised outboard ridges 32 engaging the locking screw or cap 14 (center), as compared to a conventional round stabilization rod 20 (right)—note, all pedicle screws 5 and 15 shown are monoaxial for simplicity only. In this exemplary embodiment, the stabilization rod 10 is an elongate structure that has a primarily U or circular cross-sectional shape, aside from the flat surface 12 and ridges 32 and/or 34, where the U or circular cross-sectional shape is truncated. Preferably, the stabilization rod 10 is again manufactured from a rigid or semi-rigid metallic or plastic material that is surgically compatible. The stabilization rod 10 has a minor dimension on the order to 5-10 mm and can be as long as is needed to span adjacent bone anchors in a given application. In the exemplary embodiment, the flat surface 12 engages the flat bottom of the locking screw or cap 14 as it is driven into the head body 16, thereby securing the stabilization rod 10 and preventing rotation of the stabilization rod 10 within the head body 16. Multiple flat surfaces 12 can also be used and may engage other flat surfaces of or within the head body 16. Optionally, the locking screw or cap 14, or other mating surface(s), may include corresponding ridges and/or grooves that engage the longitudinal ridges 32 and/or 34 of the stabilization rod 10, thereby further securing the stabilization rod 10 with the head body 16.

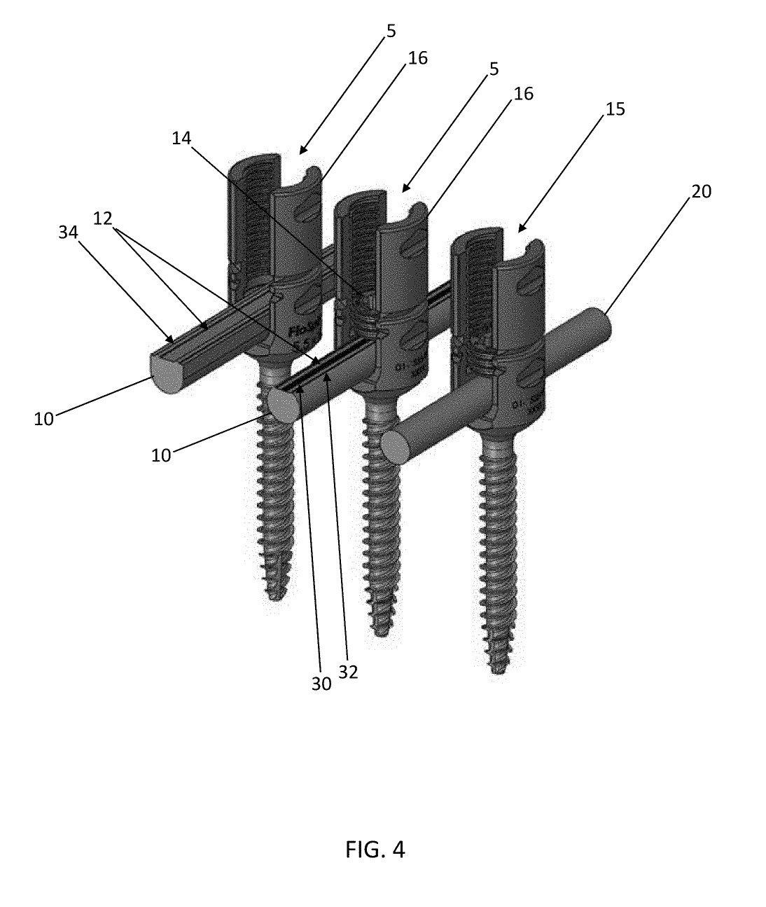

FIG. 4 is a perspective view of a still further exemplary embodiment of the flat top stabilization rod 10 of the present invention, utilizing a U-shape, one flat surface 12, and pair of raised inboard ridges 34 engaging the locking screw or cap 14 (left) and one flat surface 12, one central groove 30, and a pair of small raised outboard ridges 32 engaging the locking screw or cap 14 (center), as compared to a conventional round stabilization rod 20 (right)—note, all pedicle screws 5 and 15 shown are monoaxial for simplicity only. In this exemplary embodiment, the stabilization rod 10 is an elongate structure that has a primarily U or circular cross-sectional shape, aside from the flat surface 12, central groove 30, and/or ridges 32 and/or 34, where the U or circular cross-sectional shape is truncated. Preferably, the stabilization rod 10 is again manufactured from a rigid or semi-rigid metallic or plastic material that is surgically compatible. The stabilization rod 10 has a minor dimension on the order to 5-10 mm and can be as long as is needed to span adjacent bone anchors in a given application. In the exemplary embodiment, the flat surface 12 engages the flat bottom of the locking screw or cap 14 as it is driven into the head body 16, thereby securing the stabilization rod 10 and preventing rotation of the stabilization rod 10 within the head body 16. Multiple flat surfaces 12 can also be used and may engage other flat surfaces of or within the head body 16. Optionally, the locking screw or cap 14, or other mating surface(s), may include corresponding ridges and/or grooves that engage the longitudinal central groove 30 and/or ridges 32 and/or 34 of the stabilization rod 10, thereby further securing the stabilization rod 10 with the head body 16.

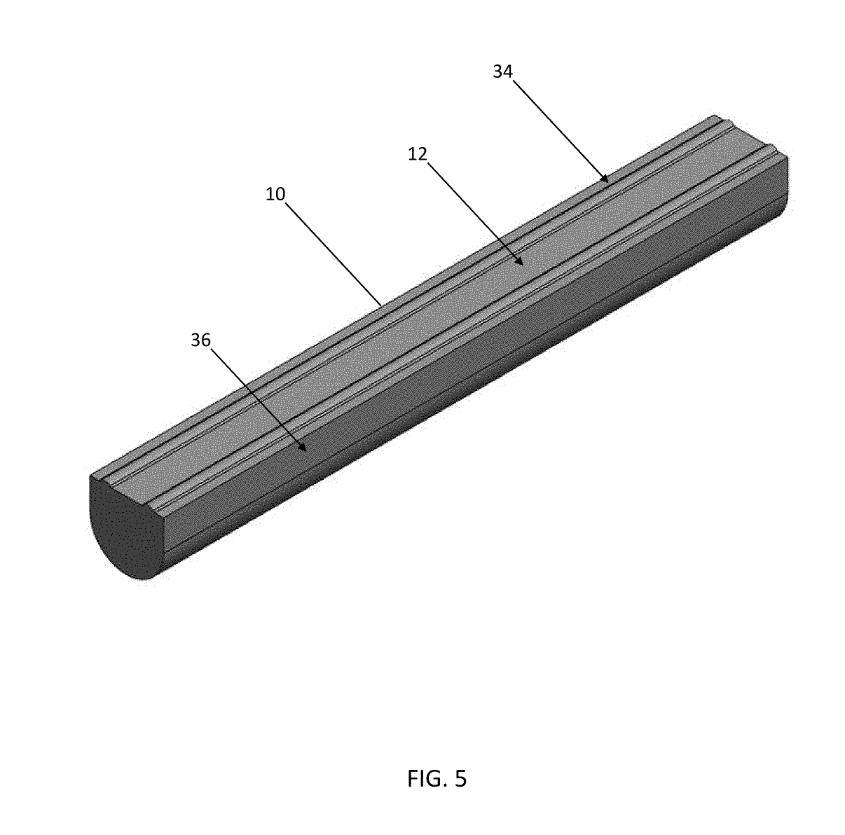

FIG. 5 is a perspective view of a still further exemplary embodiment of the flat top stabilization rod 10 of the present invention, utilizing a U-shape, one flat surface 12, and pair of raised inboard ridges 34. In this exemplary embodiment, the stabilization rod 10 is an elongate structure that has a primarily U cross-sectional shape, aside from the flat surface 12 and ridges 34, where the U cross-sectional shape is truncated. Preferably, the stabilization rod 10 is again manufactured from a rigid or semi-rigid metallic or plastic material that is surgically compatible. The stabilization rod 10 has a minor dimension on the order to 5-10 mm and can be as long as is needed to span adjacent bone anchors in a given application. In the exemplary embodiment, the flat surface 12 engages the flat bottom of the locking screw or cap (not illustrated) as it is driven into the head body (not illustrated), thereby securing the stabilization rod 10 and preventing rotation of the stabilization rod 10 within the head body. Multiple flat surfaces 12 can also be used (and are, in fact, provided by the sides 36 of the U-shaped structure) and may engage other flat surfaces of or within the head body. Optionally, the locking screw or cap, or other mating surface(s), may include corresponding grooves that engage the longitudinal ridges 34 of the stabilization rod 10, thereby further securing the stabilization rod 10 with the head body. By way of non-limiting example only, the U-shaped rod 10 measures 6 mm from side to side, for example, and 5.5 mm (or 6 mm) from top to bottom, for example. This rod 10 can only go into the head body one way, and one cannot end up with the set screw or locking cap pressing down on a corner of the flat and round surfaces, for example. The raised ridges 34 can be disposed at the outer edges of the flat surface 12 of the rod 10, or they may form a track internal to and running the length of the flat surface 12.

### LEGAL DISCLAIMER

Although the present invention is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following non-limiting claims.

What is claimed is:

1. A stabilization rod for coupling a plurality of bone anchors together, the stabilization rod comprising:
an elongate body;
wherein the elongate body comprises at least one flat surface; and
wherein the at least one flat surface is configured and positioned to engage at least one corresponding flat surface associated with one or more of a head body assembly, a set screw, and a locking cap of the bone anchor such that relative rotation of the elongate body and the bone anchor is prevented.

2. The stabilization rod of claim 1, wherein the elongate body has a substantially circular cross-sectional shape truncated by the at least one flat surface.

3. The stabilization rod of claim 1, wherein the elongate body has a substantially U-shaped cross-sectional shape truncated by the at least one flat surface.

4. The stabilization rod of claim 1, wherein the elongate body further comprises one or more longitudinal channels manufactured along the at least one flat surface.

5. The stabilization rod of claim 4, wherein the one or more of the head body assembly, the set screw, and the locking cap comprise one or more protrusions that are configured to engage the one or more longitudinal channels manufactured along the at least one flat surface such that relative rotation of the elongate body and the bone anchor is prevented.

6. The stabilization rod of claim 1, wherein the elongate body further comprises one or more longitudinal ridges manufactured along the at least one flat surface.

7. The stabilization rod of claim 6, wherein the one or more of the head body assembly, the set screw, and the locking cap comprise one or more recesses that are configured to engage the one or more longitudinal ridges manufactured along the at least one flat surface such that relative rotation of the elongate body and the bone anchor is prevented.

8. A method for providing a stabilization rod for coupling a plurality of bone anchors together, the method comprising:
providing an elongate body;
wherein the elongate body comprises at least one flat surface; and
wherein the at least one flat surface is configured and positioned to engage at least one corresponding flat surface associated with one or more of a head body assembly, a set screw, and a locking cap of the bone anchor such that relative rotation of the elongate body and the bone anchor is prevented; and
disposing the elongate body in the head body.

9. The method of claim 8, wherein the elongate body has a substantially circular cross-sectional shape truncated by the at least one flat surface.

10. The method of claim 8, wherein the elongate body has a substantially U-shaped cross-sectional shape truncated by the at least one flat surface.

11. The method of claim 8, wherein the elongate body further comprises one or more longitudinal channels manufactured along the at least one flat surface.

12. The method of claim 11, wherein the one or more of the head body assembly, the set screw, and the locking cap comprise one or more protrusions that are configured to engage the one or more longitudinal channels manufactured along the at least one flat surface such that relative rotation of the elongate body and the bone anchor is prevented.

13. The method of claim 8, wherein the elongate body further comprises one or more longitudinal ridges manufactured along the at least one flat surface.

14. The method of claim 13, wherein the one or more of the head body assembly, the set screw, and the locking cap comprise one or more recesses that are configured to engage the one or more longitudinal ridges manufactured along the at least one flat surface such that relative rotation of the elongate body and the bone anchor is prevented.

15. A bone anchor, comprising:
a threaded screw portion;
a head body coupled to the threaded screw portion; and
an elongate body coupled to the head body;
wherein the elongate body comprises at least one flat surface; and
wherein the at least one flat surface is configured and positioned to engage at least one corresponding flat surface associated with one or more of the head body assembly, a set screw, and a locking cap of the bone anchor such that relative rotation of the elongate body and the head body is prevented.

16. The bone anchor of claim 15, wherein the elongate body has a substantially circular cross-sectional shape truncated by the at least one flat surface.

17. The bone anchor of claim 15, wherein the elongate body has a substantially U-shaped cross-sectional shape truncated by the at least one flat surface.

18. The bone anchor of claim 15, wherein the elongate body further comprises one or more longitudinal channels manufactured along the at least one flat surface.

19. The bone anchor of claim 18, wherein the one or more of the head body assembly, the set screw, and the locking cap comprise one or more protrusions that are configured to engage the one or more longitudinal channels manufactured along the at least one flat surface such that relative rotation of the elongate body and the bone anchor is prevented.

20. The bone anchor of claim 15, wherein the elongate body further comprises one or more longitudinal ridges manufactured along the at least one flat surface.

21. The bone anchor of claim 20, wherein the one or more of the head body assembly, the set screw, and the locking cap comprise one or more recesses that are configured to engage the one or more longitudinal ridges manufactured along the at least one flat surface such that relative rotation of the elongate body and the bone anchor is prevented.
